(19) United States
(12) Patent Application Publication
(10) Pub. No.: US 2015/0353901 A1
(43) Pub. Date: Dec. 10, 2015

(54) Compositions and Methods for Targeted Gene Disruption in Prokaryotes

(71) Applicant: Radiant Genomics, Inc., Emeryville, CA (US)

(72) Inventors: Oliver Liu, San Francisco, CA (US); Jeffrey Kim, Berkeley, CA (US)

(73) Assignee: Radiant Genomics, Inc., Emeryville, CA (US)

(21) Appl. No.: 14/836,101

(22) Filed: Aug. 26, 2015

Related U.S. Application Data
(63) Continuation of application No. 14/537,940, filed on Nov. 11, 2014.
(60) Provisional application No. 61/902,757, filed on Nov. 11, 2013.

(51) Int. Cl. C12N 7/00

(52) U.S. Cl. CPC C12N 7/00; C12N 2795/00032

(57) ABSTRACT

The present disclosure relates to engineered bacteriophage vector compositions comprising nucleic acids that express recombinant nucleases. Also provided are methods of using engineered bacteriophage vectors to effect genomic disruption or targeted gene disruption in prokaryotes. The disclosed compositions and methods are useful for reducing antibiotic resistance in bacteria cells.

### 1. CROSS-REFERENCE TO RELATED APPLICATIONS

This application claims the benefit under 35 U.S.C. §119(e) of U.S. provisional application No. 61/902,757, filed Nov. 11, 2013, the contents of which are incorporated by reference in their entirety.

### 2. REFERENCE TO SEQUENCE LISTING

The Sequence Listing concurrently submitted herewith under 37 CFR §1.821 in a computer readable form (CRF) via EFS-Web as file name 48618US1.txt is incorporated herein by reference. The electronic copy of the Sequence Listing was created on Nov. 10, 2014, with a file size of 17,300 bytes.

### 3. BACKGROUND

The rise of antibiotic-resistant bacteria is currently one of the most serious health threats worldwide. The threat is accelerated by bacterial mechanisms for horizontal transfer of antibiotic resistance genes, coupled with selection pressures in environments that are characterized by sustained use of antibiotics, such as hospitals and in agricultural settings. Among the most pressing concerns in clinical settings are the growing prevalence of vancomycin-resistant enterococci (VRE), multi-drug resistant Staphylococcus aureus (MDRSA), and multi-drug resistant Pseudomonas aeruginosa, respectively. Enterococci are gram-positive facultatively anaerobic cocci found in a variety of environmental sources including soil, food and water, and are a common colonizing bacterial species in the human intestinal tract. Among immunocompromised patients, VRE colonization frequently leads to VRE bacteremia. Staphylococcus aureus is responsible for a variety of diseases ranging from minor skin infections to life-threatening systemic infections, including endocarditis and sepsis, and is a major cause of community- and hospital-acquired septicemia. Pseudomonas aeruginosa is a highly virulent gram-negative bacterial species that is responsible for bacteremia, wound infections, pneumonia, and urinary tract infections, and is a primary cause of pneumonia in hospitals. Also of great concern are drug resistant Enterobacter species, as well as Acinetobacter and Citrobacter (e.g., Klebsiella pneumonia).

Due to the widespread problem of antibiotic-resistant pathogenic bacteria, it has been proposed that bacteriophages be adapted for use as an alternative therapy. Bacteriophages (or phages) are viruses that naturally attack bacteria, and have been described as the most abundant biological entities on earth (Sorek et al., 2003, Annu. Rev. Biochem. 82:237). Bacteriophages have been known since the early 20^{th} century, and numerous attempts have been made since that time to develop them as antimicrobial agents. These efforts were largely unsuccessful, perhaps due to insufficient lytic activity of the bacteriophages, along with the swift rise to dominance of small molecule antibiotics. More recently, bacteriophages with greater lytic activity have been tested for therapeutic purposes (See U.S. Pat. Nos. 7,625,556, 7,674,467, and 8,282,920, and US Pat. Pub. No. 2013/0164374). However, such approaches will be vulnerable to a native bacterial adaptive immune system that has recently been shown to confer immunity against bacteriophage infection. This system is characterized by clustered, regularly interspaced short palindromic repeat (CRISPR) sequences, which are present in approximately 40% of eubacterial genomes and nearly all archaeal genomes sequenced to date. CRISPR sequences consist of short (typically 20-50 nucleotide) direct repeats separated by similarly sized, unique spacers. CRISPR loci are generally flanked by a set of CRISPR-associated (cas) protein-coding genes that are important for CRISPR maintenance and function. In Streptococcus thermophilus and Escherichia coli, CRISPR/cas loci have recently been demonstrated to act by an interference mechanism that relies on the sequence identity between CRISPR spacers and foreign DNA sequences.

As bacteria evolve to develop resistance to various antimicrobial agents such as small molecule antibiotics and bacteriophages, there is a clear need for new drug agents that efficiently address the threat of bacterial antibiotic resistance by specifically targeting and disrupting antibiotic resistance genes.

### 4. SUMMARY

The present disclosure provides compositions comprising bacteriophages that are useful for the targeted disruption of genes within prokaryotic cells. The disclosed methods provide for “immunization” of bacteria against the introduction of undesirable genetic material such as drug resistance genes and genes associated with toxicity or pathogenicity.

In one aspect, disclosed bacteriophages are used to disrupt the genomes and thereby inhibit the growth and proliferation of bacterial cells. In another aspect, the disclosed bacteriophages are used to target and disrupt drug resistance genes that are found on plasmids or integrated into the genomic DNA within bacterial cells. The disclosed methods have numerous advantages over existing approaches like small molecule or protein-based antibiotics, including the ability to target very specific strains of bacteria while leaving other strains unaffected. Thus pathogenic bacteria can be targeted in the gut, for example, without harming commensal or symbiotic (“good” bacteria) strains that reside in digestive tract or on the skin and benefit their human hosts. The group of target strains can be broadened or narrowed according to purpose by using mixtures of bacteriophages. Mixtures of bacteriophages using different mechanisms of infection also serve to defeat the evolution of resistance. By targeting multiple sequences (e.g. by using a CRISPR-array), the disclosed methods also provide a mechanism to avoid the development of resistance through single nucleotide polymorphisms (SNPs) in single locations. The likelihood that an organism develops multiple SNPs to avoid CRISPR/Cas9 targeting is much lower when using multiple guide sequences.

Through specificity of targeting, the disclosed methods can be used to specifically modulate complex microbiomes through the targeting of specific species in complex microbial communities. For example, even if a bacteriophage transduces a wide range of species in a microbiome, the targeting sequences can be tuned to specific genetic signatures found only in certain species.

The bacteriophage-based platforms described herein are useful for targeted delivery of nucleases (e.g., CRISPR-Cas9 system, TALENs, zinc finger nucleases). Bacteriophage can efficiently deliver a system to a wide range of compatible bacteria in order to affect survival, virulence, the uptake of other plasmids/bacteriophage, or any number of pathways and genetic subsystems in the target organisms. In some embodiments, the bacteriophage is non-lytic and does not affect transduced organisms if no compatible guide sequences are present. In some embodiments, by using compatible guide sequences engineered into the delivery plasmid, non-lytic bacteriophage can kill, modify, or immunize a transduced host cell.

The disclosure provides methods of treatment for humans and other animals, involving delivery of disclosed bacteriophage compositions. The compositions can also be applied to a variety of surfaces as prophylactic measures against biofilm formation for pathogenic organisms, or used in aerosol form for agricultural applications, where the development of antibiotic resistance can be of particular concern.

Accordingly, in some embodiments, disclosed bacteriophages comprise a polynucleotide that expresses a polypeptide comprising: (a) a targeting module; and (b) a nuclease module, wherein the targeting module tethers the nuclease module to a target DNA sequence within a prokaryotic host cell. The targeting module can be covalently linked to the nuclease module, or the targeting module may be noncovalently bound to the nuclease module.

### 5. BRIEF DESCRIPTION OF THE FIGURES

FIG. 1 provides a schematic of a polynucleotide useful for targeted gene disruption as disclosed.

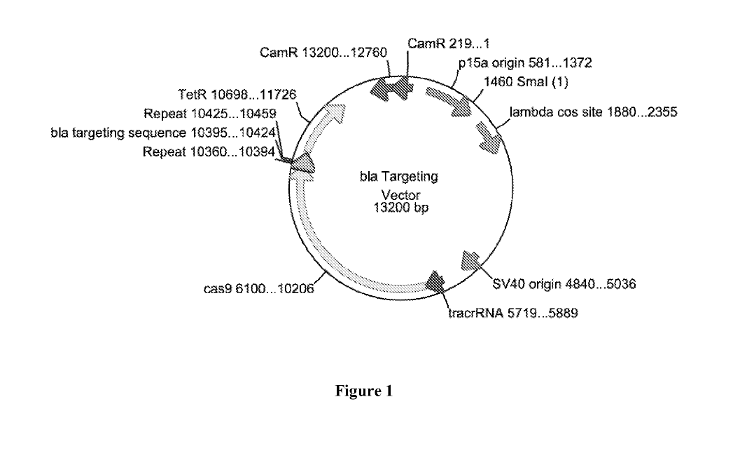

FIG. 2 provides data showing targeted disruption of antibiotic resistance using an RNA-directed DNA-binding polypeptide of the disclosure. Plasmids contained either an ampicillin resistance gene (plasmid 1) or a kanamycin resistance gene (plasmid 2), and were contacted with constructs directed by guide RNAs to target plasmids 1 and 2 (gRNA 1), plasmid 2 alone (gRNA 2), or a nonhomologous sequence (gRNA 3)

### 6. DETAILED DESCRIPTION

As used herein and in the appended claims, the singular forms “a,” “and,” and “the” include plural referents unless the context clearly dictates otherwise. Thus, for example, reference to “a polynucleotide” includes a plurality of such polynucleotides and reference to “the microorganism” includes reference to one or more microorganisms, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

6.1. Bacteriophages

Bacteriophages are viruses that infect bacteria. Bacteriophages have traditionally been used in their lytic state in order to kill bacteria by lysis, an approach that relies on strong lytic activity and high doses of the bacteriophages, and has been only modestly effective in clinical contexts. In an alternative approach, there have been a few reports of studies using genetically engineered bacteriophages in the non-lytic state as vehicles for the delivery of antimicrobial agents to bacteria. In this way, rather than to kill bacteria as they do in nature by lysis, the engineered bacteriophages integrate genes into the bacteria that result in lethality, restricted growth and proliferation, or reduced virulence.

In one study, bacteriophages were engineered to carry one or more genes encoding bactericidal proteins that act in a bacterial programmed cell death system. These engineered bacteriophages were shown to reduce bacterial numbers in vitro and in a bacteremic mouse model of infection. See Westwater et al., 2003, Antimicrob. Agents Chemother. 47(4):1301. Bacteriophages have also been used to deliver photosensitizing agents to bacteria associated with topical infections. These bacteriophages were found effective in treating topical infections in combination with light therapy to activate the photosensitizing agents. See Embleton et al., 2005, Antimicrob Agents Chemother. 49(9):3690. Use of bacteriophages to overexpress lexA3 and thereby suppress the SOS network in E. coli was also successful in sensitizing these bacterial cells to treatment with quinolones and other antibiotics, and it has been suggested that bacteriophages engineered to overexpress certain regulatory proteins can serve as effective adjuvants for antibiotic treatment of bacterial infections. A very similar approach is described in US Pat. Pub. No. 2010/0322903

Bacteriophages have also been used to deliver restriction endonucleases to kill host cells. In one study, an export protein gene of the P. aeruginosa filamentous bacteriophage Pf3 was replaced with a restriction endonuclease gene, resulting in killing of P. aeruginosa host cells and increased survival of infected mice. See Hagens et al., 2004, Antimicrob. Agents Chemother. 48(10):3817. Restriction endonucleases, also known as restriction enzymes, cut DNA at specific recognition nucleotide sequences known as restriction sites. Because these sites are small, typically between 4 and 8 nucleotides in length, restriction enzymes are not very specific in the context of a genome.

Bacteriophages of the disclosure can be any bacteriophage that infects a host cell as described in Section 6.2, below. In some embodiments, the host cell is an Escherichia coli (E. coli) cell, and the bacteriophage is a lambda phage.

Methods of isolating bacteriophages from bacterial hosts of various species have been described. See, e.g., Clokie and Kropinski (eds.), 2009, Bacteriophages: Methods and Protocols, Vol. 1, Chapter 3.

6.2. Host Cells

Naturally occurring bacteriophages outnumber their bacterial prey in both numbers and diversity. Accordingly, bacteriophages of the disclosure are capable of binding to and introducing genetic material into host cells of a multitude of species including, for example, Gram negative or Gram positive bacteria. Non-limiting examples of Gram negative bacteria include Escherichia coli, Klebsiella spp. (typically K. pneumoniae or K. oxytoca), Enterobacter spp (typically E. cloacae or E. aerogenes), Bordetella spp. (typically B. bronchiseptica, B. pertussis or B. parapertussis), Chlamydia spp. (typically C. trachomatis), Legionella spp. (typically L. neumophilia), Pseudemonas spp. (typically P. aeruginosa), Mycoplasma spp. (typically M. pneumoniae), Haemophilus influenza, Serratia marcescens, Proteus mirabilis, Aninetobacter baumannii, Stenotrophomonas maltophilia and Neisseria meningitidis (typically of serogroup A, B, C, H, I, K, L, X, Y, Z, 29E or W135). Non-limiting examples of Gram positive bacteria include Staphylococcus spp. (typically S. aureus or Coagulase negative Staphylococci), Streptococcus spp. (typically S. pneumoniae or S. pyogenes) and Enterococcus spp. (typically E. faecium or E. faecalis).

In some embodiments, a host cell is of a species selected from: Staphylococcus aureus, Staphylococcus epidermidis, Helicobacter pylori, Streptococcus pneumoniae, Streptococcus mutans, Streptococcus oralis, Streptococcus parasanguis, Streptococcus pyogenes, Streptococcus viridans, Group A streptococcus and anaerobic streptococcus, Hemophilus influenzae, Shigella dysenteriae, Mycobacterium tuberculosis, Mycobacterium leprae, Mycobacterium asiaticum, Mycobacterium intracellulare, Mycoplasma pneumoniae, Mycoplasma hominis, Neisseria meningitidis, Neisseria gonorrhea, Klebsiella pneumoniae, Pseudomonas aeruginosa, Propionibacterium acnes, Treponema pallidum, Treponema pertanue, Treponema carateum, Escherichia coli, Salmonella typhimurium, Borrelia burgdorferi, and Leptospirex.

In some embodiments, a host cell is of a pathogenic species selected from: Acinetobacter baumannii, Enterococcus faecalis, Enterococcus faecium, Pseudomonas aeruginosa, Staphylococcus aureus, Staphylococcus epidermidis, Staphylococcus auricularis, Staphylococcus capitis, Staphylococcus haemolyticus, Staphylococcus hominis, Staphylococcus saprophyticus, Staphylococcus simulans, Staphylococcus xylosis, Micrococcus luteus, Bacillus subtilis, Bacillus pumilus, Enterococcus hirae and Enterococcus avium.

The method described herein can be applied to any species of bacteria that produces an RNA involved in conferring drug resistance on the bacteria. The disclosed method can be used to convert the phenotype of drug-resistant bacteria to a drug-sensitive phenotype in any setting. Preferred target bacteria are those that infect, colonize, or otherwise grow in or on plants or animals, including humans. Preferred target bacteria can harbor transmissible plasmids, episomal vectors, or viral vectors that integrate into the host chromosome with a high frequency which carry drug resistance markers. RNAs can confer resistance to any known antimicrobial agent including, but not limited to aminoglycosides, cephalosporins, fluoroquinolones, lincosamides, macrolides, penicillins, sulfonamides, or tetracyclins. Thus, for example, suitable antibiotics include, but are not limited to, Gentamicin, Kanamycin, Neomycin, Streptomycin, Tobramycin, Cefazolin, Cephalexin, Cephapirin, Cephradine, Cefuroxime, Cefixime, Cefotaxime, Ceftazidime, Ceftizoxime, Ceftriaxone, Ciprofloxacin, Levofloxacin, Ofloxacin, Clindamycin, Azithromycin, Clarithromycin, Erythromycin, Amoxicillin, Ampicillin, Ampicillin-Sulbactam, Cloxacillin, Dicloxacillin, Mezlocillin, Methicillin, Nafcillin, Oxacillin, Penicillin G Benzathine, Penicillin G Potassium, Penicillin G Procaine, Penicillin V Potassium, Piperacillin, Ticarcillin, Ticarcillin-Clavulanate potassium, Pyrimethamine-Sulfadoxine, Sulfadizine, Sulfisoxazole, Sulfrnethoxazole, Trimethoprim-sulfamethoxazole, Chlortetracycline, Doxycycline, and Tetracycline.

In some embodiments, a host cell is a Staphylococcus aureus bacterium that has developed antibiotic resistance to all penicillins, including methicillin, referred to as methicillin-resistant Staphylococcus aureus (MRSA). In some embodiments, a host cell is a vancomycin intermediately sensitive Staphylococcus aureus (VISA), vancomycin resistant Staphylococcus aureus (VRSA) and vancomycin resistant Enterococci (VRE).

VISA, VRSA and VRE are genotypically and phenotypically distinct from vancomycin sensitive gram-positive bacteria, tending to form discrete clonal lineages. The acquisition of mobile genetic elements carrying resistance genes and often virulence determinants results in strains that are often resistant to a number of drugs. Resistance can be specific, i.e. particular to a certain drug or class of drugs or non-specific in that the resistance applies to a range of drugs, not necessarily related. In the case of VISA, an increase in cell wall thickness is a major contributor to the observed drug resistance.

VISA, VRSA and VRE may be defined as any staphylococcal or enterococcal strain with a vancomycin MIC of 4-8 mg/L (VISA) or greater or equal to 8 mg/L (VRSA and VRE). These levels of resistance may be due to an increase in cell wall thickness, by the production of cell-wall precursors incapable of binding vancomycin, or via another mechanism. Susceptible gram-positive organisms synthesise cell wall precursors ending in D-ala-D-ala, whereas vancomycin resistant gram-positive organisms synthesize, for example, D-ala-D-lac precursors. The presence of vancomycin resistance in staphylococcal or enterococcal strains may be identified by the measurement of the MIC to vancomycin by broth or agar dilution, or by Etest®, or by the identification of vanA, vanB, vanC, vanD, vanE, vanG genes, or similar, by polymerases chain reaction (PCR). The current disclosure also encompasses the subclass of VISA strains that are heterogeneous VISA (hVISA); these are vancomycin susceptible methicillin-resistant Staphylococcus aureus by conventional testing but have a subpopulation of intermediately resistant cells.

In some embodiments, preferred target bacterial cells are those that colonize, infect, or otherwise grow in or on animals. Particularly preferred are bacterial cells that colonize, infect, or grow in or on skin, in the gastrointestinal tract or in the respiratory tract. Also preferred are bacterial cells that colonize, infect, or grow in the urogenital tract. Some preferred bacterial cells belong to one of the families Enterobacteriaceae, Micrococcaceae, Vibrionaceae, Pasteurellaceae, Mycoplasmataceae, or Rickettsiaceae. Within these families, preferred bacterial cells belong to one of the genera Salmonella, Shigella, Escherichia, Enterobacter, Serratia, Proteus, Yersinia, Citrobacter, Edwardsiella, Providencia, Klebsiella, Hafnia, Ewingella, Kluyvera, Morganella, Planococcus, Stomatococcus, Micrococcus, Staphylococcus, Vibrio, Aeromonas, Plessiomonas, Haemophilus, Actinobacillus, Pasteurella, Mycoplasma, Ureaplasma, Rickettsia, Coxiella, Rochalimaea, or Ehrlichia. Particular preferred bacterial cells are those that belong to the family Enterobacteriaceae.

Preferred bacterial cells belong to one of the genera Salmonella, Shigella, Escherichia, Enterobacter, Serratia, Proteus, Yersinia, Citrobacter, Edwardsiella, Providencia, Klebsiella, Hafnia, Ewingella, Kluyvera, Morganella, Planococcus, Stomatococcus, Micrococcus, Staphylococcus, Vibrio, Aeromonas, Plessiomonas, Haemophilus, Actinobacillus, Pasteurella, Mycoplasma, Ureaplasma, Rickettsia, Coxiella, Rochalimaea, Ehrlichia, Streptococcus, Enterococcus, Aerococcus, Gemella, Lactococcus, Leuconostoc, Pedicoccus, Bacillus, Corynebacterium, Arcanobacterium, Actinomyces, Rhodococcus, Listeria, Erysipelothrix, Gardnerella, Neisseria, Camylobacter, Arcobacter, Wolinella, Heliobacter, Achomobacter, Acinetobacter, Agrobacterium, Alcaligenes, Chryseomonas, Comamonas, Eikenella, Flavimonas, Flavobacterium, Moraxella, Oligella, Pseudomonas, Skewanella, Weeksella, Xanthomonas, Bordetella, Franciesella, Brucella, Legionella, Afipia, Bartonella, Calymmatobacterium, Cardiobacterium, Streptobacillus, Spirillum, Peptostreptococcus, Peptococcus, Sarcinia, Coprococcus, Ruminococcus, Propionibacterium, Mobiluncus, Bifidobacterium, Eubacterium, Lactobacillus, Rothia, Clostridium, Bacteroides, Porphyromonas, Prevotella, Fusobacterium, Bilophila, Leptotrichia, Wolinella, Acidaminococcus, Megasphaera, Veilonella, Norcardia, Actinomadura, Norcardiopsis, Streptomyces, Micropolysporas, Thermoactinomycetes, Mycobacterium, Treponema, Borrelia, Leptospira, or Chlamydiae.

6.3. Target DNA Sequences

The disclosed bacteriophages are engineered to express polypeptides that comprise tethering modules that bind specifically to target DNA sequences. A target DNA sequence can be any specific DNA sequence in a host cell, whether on a plasmid or a host cell chromosome. In some embodiments, a target DNA sequence is a sequence that is specific to a particular bacterial species, or to a particular strain of bacteria.

In some embodiments, a target DNA sequence is within a gene that confers resistance to a drug agent. The gene that confers resistance to a drug agent can be located in a plasmid or in the genomic DNA of the bacteria, also referred to as the bacterial chromosome. In some embodiments, a target DNA sequence is within a gene regulatory region that regulates a gene that confers resistance to a drug agent. Gene regulatory regions are known and include, but are not limited to, promoters, enhancers, transcriptional terminators. A gene regulatory region also includes any portion of a DNA sequence within a host cell that influences the activity of a gene or gene product, directly or indirectly, e.g., by increasing or decreasing transcription of the gene, or by affecting the stability of the gene transcript, or by modulating translation of the gene product.

In some embodiments, a target DNA sequence is selected from any of the sequences listed in Table 1, below.

TABLE 1

| SEQ | | |
|---|---|---|
| ID NO: | Description | Sequence |
| n/a | E. coli | 5′-NNNNNNNNNNNN-AWG-3 |
| n/a | SpCas9 PAM | 5′-NNNNNNNNNNNN-NGG-3 |
| | (S. pyogenes | |
| | protospacer | |
| | adjacent motif) | |
| n/a | H. solfataricus | 5′-NNNNNNNNNNNN-CCN-3 |
| n/a | H. solfataricus | 5′-NNNNNNNNNNNN-TCN-3 |
| n/a | H. walsbyi | 5′-NNNNNNNNNNNN-TTC-3 |
| n/a | P. aeruginosa | 5′-NNNNNNNNNNNN-CC-3 |
| n/a | S. thermophilus | 5′-NNNNNNNNNNNN-NNAGAAW-3 |
| n/a | S. thermophilus | 5′-NNNNNNNNNNNN-NGGNG-3 |
| n/a | N. meningititus | 5′-NNNNNNNNNNNN-GATT-3 |

6.4. Targeting Modules

Polypeptides of the disclosure specifically bind target DNA sequences via targeting modules. In this way, a targeting module is characterized by its ability to recognize and bind to a target DNA sequence in a sequence-specific manner. Sequence-specific polypeptide DNA-binding domains are known. Their sequence specificity is conferred by the three-dimensional structure of the folded polypeptide which allows it to make energetically favorable contacts to a specific sequence of nucleotides.

One class of well-characterized sequence-specific polypeptide DNA-binding domains are the zinc fingers. Zinc fingers are well-known small polypeptide structures that conjugate zinc ions and bind to DNA in a sequence-specific manner.

A second class of known sequence-specific nucleases are the transcription activator-like effector nucleases, or TALENs. These recombinant enzymes are created by fusing a TAL effector DNA binding domain to a nuclease domain. TAL effectors are produced by Xanthomonas bacteria and are characterized by sequence-specific DNA binding domains. The specificity is conferred by a variable pair of amino acids within a repeating sequence of 33-34 amino acids. Methods for designing and generating TALENs are known (see, e.g., US Pat. Pub. No. 2013/0117869).

A third class of sequence-specific polypeptide DNA-binding domains are RNA-directed DNA-binding domains. These polypeptides do not exhibit sequence-specific DNA binding on their own, but bind to nucleic acid molecules (e.g., RNA) that guide the polynucleotide to a target DNA sequence through base pairing. These RNAs are sometimes referred to as “guide RNAs,” and are complementary or nearly complementary in sequence to their target DNA sequences.

Thus, a targeting module can be an RNA-directed DNA-binding polypeptide. Such targeting modules are characterized by a cleft within the polynucleotide that serves as a binding site for a portion of a guide RNA. Another portion of the guide RNA then binds to a target DNA sequence via base pairing, allowing the guide RNA to tether the targeting module to a target DNA sequence. Because it relies on a guide RNA for its targeting function, a targeting module that is an RNA-directed DNA-binding polypeptide should be co-expressed with the guide RNA.

RNA-directed DNA-binding polypeptides are advantageous because they have sequence specificity given by the length of the guide RNA. The regions of complementarity between guide RNA and target are frequently much longer than the sequences recognized by, for example, restriction endonucleases which are typically between 4-8 nucleotides. Accordingly, the present disclosure provides for targeting modules recognizing target DNA sequences of lengths greater than 10 nucleotides, greater than 11 nucleotides, greater than 12 nucleotides, greater than 13 nucleotides, greater than 14 nucleotides, greater than 15 nucleotides, greater than 16 nucleotides, greater than 17 nucleotides, greater than 18 nucleotides, greater than 19 nucleotides, greater than 20 nucleotides, greater than 21 nucleotides, greater than 22 nucleotides, greater than 23 nucleotides, greater than 24 nucleotides, greater than 25 nucleotides, greater than 26 nucleotides, greater than 27 nucleotides, greater than 28 nucleotides, greater than 29 nucleotides, greater than 30 nucleotides, greater than 32 nucleotides, and greater than 35 nucleotides. Skilled artisans will understand that, in order to provide sequence specific targeting in this context, a target DNA sequence will not need to be longer than at most 100 nucleotides.

The targeting module can be a naturally occurring polypeptide or fragment thereof, or it can be a recombinant polypeptide. Similarly, a guide RNA can be a naturally occurring polynucleotide or fragment thereof. Recombinant RNA-directed DNA-binding polypeptide and recombinant guide RNAs based on components of the CRISPR system have been described. In particular, recombinant Cas9 has been co-expressed with a guide RNA to allow its sequence-specific recruitment to a target DNA sequence, determined by complementarity to the guide RNA. See Qi et al., 2013, Cell, 152(5):1173 and Gilbert et al., 2013, Cell, 154(2):442. Skilled artisans will appreciate that any of the three types of Cas systems, Types I, II, and III, can be employed in the disclosed methods (see Westra et al., 2012, Annu Rev Genet. 46:311 and Sorek et al., 2013, Annu Rev Biochem. 82:237).

Thus in some embodiments, the targeting module comprises a guide RNA. One exemplary embodiment of a guide RNA is provided in Table 2, below.

TABLE 2

| SEQ | | |
|---|---|---|
| ID NO: | Description | Sequence |
| 1 | E. coli | 5′-aaaccgcctccatccagtctatt |
| | ampicillin | aattgttgccg-3′ |
| | resistance | |
| | guide RNA | |

6.5. Nuclease Modules

Nucleases, sometimes referred to as “polynucleotidases” or “nucleodepolymerases” are enzymes that are capable of cleaving the phosphodiester bonds between the nucleotide subunits of nucleic acids. Nucleases are usually further divided into endonucleases and exonucleases, although some enzymes may fall in both categories.

In some embodiments, the nuclease module comprises a component of the CRISPR system, e.g., Cas9.

The present disclosure also provides for sequences with greater than 97% identity, greater than 95% identity, greater than 90% identity, greater than 85% identity, greater than 80% identity, greater than 75% identity, greater than 70% identity, greater than 65% identity, greater than 60% identity, greater than 55% identity, or greater than 50% identity compared to SEQ ID NO:2 given in Table 3, below.

TABLE 3

| SEQ | | |
|---|---|---|
| ID | Descrip- | |
| NO: | tion | Sequence |
| 2 | Cas9 | MDKKYSIGLDIGTNSVGWAVITDEYKVPSKKFKVLGNTDR |
| | protein | HSIKKNLIGALLFDSGETAEATRLKRTARRRYTRRKNRIC |
| | sequence | YLQEIFSNEMAKVDDSFFHRLEESFLVEEDKKHERHPIFG |
| | | NIVDEVAYHEKYPTIYHLRKKLVDSTDKADLRLIYLALAH |
| | | MIKFRGHFLIEGDLNPDNSDVDKLFIQLVQTYNQLFEENP |
| | | INASGVDAKAILSARLSKSRRLENLIAQLPGEKKNGLFGN |
| | | LIALSLGLTPNFKSNFDLAEDAKLQLSKDTYDDDLDNLLA |
| | | QIGDQYADLFLAAKNLSDAILLSDILRVNTEITKAPLSAS |
| | | MIKRYDEHHQDLTLLKALVRQQLPEKYKEIFFDQSKNGYA |
| | | GYIDGGASQEEFYKFIKPILEKMDGTEELLVKLNREDLLR |
| | | KQRTFDNGSIPHQIHLGELHAILRRQEDFYPFLKDNREKI |
| | | EKILTFRIPYYVGPLARGNSRFAWMTRKSEETITPWNFEE |
| | | VVDKGASAQSFIERMTNFDKNLPNEKVLPKHSLLYEYFTV |
| | | YNELTKVKYVTEGMRKPAFLSGEQKKAIVDLLFKTNRKVT |
| | | VKQLKEDYFKKIECFDSVEISGVEDRFNASLGTYHDLLKI |
| | | IKDKDFLDNEENEDILEDIVLTLTLFEDREMIEERLKTYA |
| | | HLFDDKVMKQLKRRRYTGWGRLSRKLINGIRDKQSGKTIL |
| | | DFLKSDGFANRNFMQLIHDDSLTFKEDIQKAQVSGQGDSL |
| | | HEHIANLAGSPAIKKGILQTVKVVDELVKVMGRHKPENIV |
| | | IEMARENQTTQKGQKNSRERMKRIEEGIKELGSQILKEHP |
| | | VENTQLQNEKLYLYYLQNGRDMYVDQELDINRLSDYDVDH |
| | | IVPQSFLKDDSIDNKVLTRSDKNRGKSDNVPSEEVVKKMK |
| | | NYWRQLLNAKLITQRKFDNLTKAERGGLSELDKAGFIKRQ |
| | | LVETRQITKHVAQILDSRMNTKYDENDKLIREVKVITLKS |
| | | KLVSDFRKDFQFYKVREINNYHHAHDAYLNAVVGTALIKK |
| | | YPKLESEFVYGDYKVYDVRKMIAKSEQEIGKATAKYFFYS |
| | | NIMNFFKTEITLANGEIRKRPLIETNGETGEIVWDKGRDF |
| | | ATVRKVLSMPQVNIVKKTEVQTGGFSKESILPKRNSDKLI |
| | | ARKKDWDPKKYGGFDSPTVAYSVLVVAKVEKGKSKKLKSV |
| | | KELLGITIMERSSFEKNPIDFLEAKGYKEVKKDLIIKLPK |
| | | YSLFELENGRKRMLASAGELQKGNELALPSKYVNFLYLAS |
| | | HYEKLKGSPEDNEQKQLFVEQHKHYLDEIIEQISEFSKRV |
| | | ILADANLDKVLSAYNKHRDKPIREQAENIIHLFTLTNLGA |
| | | PAAFKYFDTTIDRKRYTSTKEVLDATLIHQSITGLYETRI |
| | | DLSQLGGD* |
| 3 | Cas 9 | atggataagaaatactcaataggcttagatatcggcacaa |
| | nucleic | atagcgtcggatgggcggtgatcactgatgaatataaggt |
| | acid | tccgtctaaaaagttcaaggttctgggaaatacagaccgc |
| | Sequence | cacagtatcaaaaaaaatcttataggggctcttttatttg |
| | | acagtggagagacagcggaagcgactcgtctcaaacggac |
| | | agctcgtagaaggtatacacgtcggaagaatcgtatttgt |
| | | tatctacaggagattttttcaaatgagatggcgaaagtag |
| | | atgatagtttattcatcgacttgaagagtcttttttggtg |
| | | gaagaagacaagaagcatgaacgtcatcctatttttggaa |
| | | atatagtagatgaagttgcttatcatgagaaatatccaac |
| | | tatctatcatctgcgaaaaaaattggtagattctactgat |
| | | aaagcggatttgcgcttaatctatttggccttagcgcata |
| | | tgattaagtttcgtggtcattttttgattgagggagattt |
| | | aaatcctgataatagtgatgtggacaaactatttatccag |
| | | ttggtacaaacctacaatcaattatttgaagaaaacccta |
| | | ttaacgcaagtggagtagatgctaaagcgattctttctgc |
| | | acgattgagtaaatcaagacgattagaaaatctcattgct |
| | | cagctccccggtgagaagaaaaatggcttatttgggaatc |
| | | tcattgctttgtcattgggtttgacccctaattttaaatc |
| | | aaattttgatttggcagaagatgctaaattacagctttca |
| | | aaagatacttacgatgatgatttagataatttattggcgc |
| | | aaattggagatcaatatgctgatttgtttttggcagctaa |
| | | gaatttatcagatgctattttactttcagatatcctaaga |
| | | gtaaatactgaaataactaaggctcccctatcagcttcaa |
| | | tgattaaacgctacgatgaacatcatcaagacttgactct |
| | | tttaaaagctttagttcgacaacaacttccagaaaagtat |
| | | aaagaaatcttttttgatcaatcaaaaaacggatatgcag |
| | | gttatattgatgggggagctagccaagaagaattttataa |
| | | atttatcaaaccaattttagaaaaaatggatggtactgag |
| | | gaattattggtgaaactaaatcgtgaagatttgctgcgca |
| | | agcaacggacctttgacaacggctctattccccatcaaat |
| | | tcacttgggtgagctgcatgctattttgagaagacaagaa |
| | | gacttttatccatttttaaaagacaatcgtgagaagattg |
| | | aaaaaatcttgacttttcgaattccttattatgttggtcc |
| | | attggcgcgtggcaatagtcgttttgcatggatgactcgg |
| | | aagtctgaagaaacaattaccccatggaattttgaagaag |
| | | ttgtcgataaaggtgcttcagctcaatcatttattgaacg |
| | | catgacaaactttgataaaaatcttccaaatgaaaaagta |
| | | ctaccaaaacatagtttgctttatgagtattttacggttt |
| | | ataacgaattgacaaaggtcaaatatgttactgaaggaat |
| | | gcgaaaaccagcatttattcaggtgaacagaagaaagcca |
| | | ttgttgatttactcttcaaaacaaatcgaaaagtaaccgt |
| | | taagcaattaaaagaagattatttcaaaaaaatagaatgt |
| | | tttgatagtgttgaaatttcaggagttgaagatagattta |
| | | atgcttcattaggtacctaccatgatttgctaaaaattat |
| | | taaagataaagattttttggataatgaagaaaatgaagat |
| | | atcttagaggatattgttttaacattgaccttatttgaag |
| | | atagggagatgattgaggaaagacttaaaacatatgctca |
| | | cctattgatgataaggtgatgaaacagcttaaacgtcgcc |
| | | gttatactggttggggacgtttgtctcgaaaattgattaa |
| | | tggtattagggataagcaatctggcaaaacaatattagat |
| | | tttttgaaatcagatggttttgccaatcgcaattttatgc |
| | | agctgatccatgatgatagtttgacatttaaagaagacat |
| | | tcaaaaagcacaagtgtctggacaaggcgatagtttacat |
| | | gaacatattgcaaatttagctggtagccctgctattaaaa |
| | | aaggtattttacagactgtaaaagttgttgatgaattggt |
| | | caaagtaatggggcggcataagccagaaaatatcgttatt |
| | | gaaatggcacgtgaaaatcagacaactcaaaagggccaga |
| | | aaaattcgcgagagcgtatgaaacgaatcgaagaaggtat |
| | | caaagaattaggaagtcagattcttaaagagcatcctgtt |
| | | gaaaatactcaattgcaaaatgaaaagctctatctctatt |
| | | atctccaaaatggaagagacatgtatgtggaccaagaatt |
| | | agatattaatcgtttaagtgattatgatgtcgatcacatt |
| | | gttccacaaagtttccttaaagacgattcaatagacaata |
| | | aggtcttaacgcgttctgataaaaatcgtggtaaatcgga |
| | | taacgttccaagtgaagaagtagtcaaaaagatgaaaaac |
| | | tattggagacaacttctaaacgccaagttaatcactcaac |
| | | gtaagtttgataatttaacgaaagctgaacgtggaggttt |
| | | gagtgaacttgataaagctggttttatcaaacgccaattg |
| | | gttgaaactcgccaaatcactaagcatgtggcacaaattt |
| | | tggatagtcgcatgaatactaaatacgatgaaaatgataa |
| | | acttattcgagaggttaaagtgattaccttaaaatctaaa |
| | | ttagtttctgacttccgaaaagatttccaattctataaag |
| | | tacgtgagattaacaattaccatcatgcccatgatgcgta |
| | | tctaaatgccgtcgttggaactgctttgattaagaaatat |
| | | ccaaaacttgaatcggagtttgtctatggtgattataaag |
| | | tttatgatgttcgtaaaatgattgctaagtctgagcaaga |
| | | aataggcaaagcaaccgcaaaatatttatttactctaata |
| | | tcatgaacttcttcaaaacagaaattacacttgcaaatgg |
| | | agagattcgcaaacgccctctaatcgaaactaatggggaa |
| | | actggagaaattgtctgggataaagggcgagattttgcca |
| | | cagtgcgcaaagtattgtccatgccccaagtcaatattgt |
| | | caagaaaacagaagtacagacaggcggattctccaaggag |
| | | tcaattttaccaaaaagaaattcggacaagcttattgctc |
| | | gtaaaaaagactgggatccaaaaaaatatggtggttttga |
| | | tagtccaacggtagcttattcagtcctagtggttgctaag |
| | | gtggaaaaagggaaatcgaagaagttaaaatccgttaaag |
| | | agttactagggatcacaattatggaaagaagttcctttga |
| | | aaaaaatccgattgaattttagaagctaaaggatataagg |
| | | aagttaaaaaagacttaatcattaaactacctaaatatag |
| | | tattttgagttagaaaacggtcgtaaacggatgctggcta |
| | | gtgccggagaattacaaaaaggaaatgagctggctctgcc |
| | | aagcaaatatgtgaattttttatatttagctagtcattat |
| | | gaaaagttgaagggtagtccagaagataacgaacaaaaac |
| | | aattgtttgtggagcagcataagcattatttagatgagat |
| | | tattgagcaaatcagtgaattttctaagcgtgttatttta |
| | | gcagatgccaatttagataaagttcttagtgcatataaca |
| | | aacatagagacaaaccaatacgtgaacaagcagaaaatat |
| | | tattcatttatttacgttgacgaatcttggagctcccgct |
| | | gatttaaatattttgatacaacaattgatcgtaaacgata |
| | | tacgtctacaaaagaagttttagatgccactcttatccat |
| | | caatccatcactggtctttatgaaacacgcattgatttga |
| | | gtcagctaggaggtgactga |

Also provided are sequences hybridizable to a sequence complementary to Cas9, e.g., to Cas9 mRNA.

A nucleic acid molecule is “hybridizable” to another nucleic acid molecule, such as a cDNA, genomic DNA, or RNA molecule, when a single stranded form of the nucleic acid molecule can anneal to the other nucleic acid molecule under the appropriate conditions of temperature and solution ionic strength. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein (entirely incorporated herein by reference). The conditions of temperature and ionic strength determine the “stringency” of the hybridization. Stringency conditions can be adjusted to screen for moderately similar fragments (such as homologous sequences from distantly related organisms), to highly similar fragments (such as genes that duplicate functional enzymes from closely related organisms). Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS is increased to 60° C. Another preferred set of highly stringent conditions uses two final washes in 0.1×SSC, 0.1% SDS at 65° C. An additional set of stringent conditions include hybridization at 0.1×SSC, 0.1% SDS, 65° C. and washed with 2×SSC, 0.1% SDS followed by 0.1×SSC, 0.1% SDS, for example.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least 30 nucleotides. Furthermore, the skilled artisan will recognize that the temperature and wash solution salt concentration may be adjusted as necessary according to factors such as length of the probe.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignment of the sequences may be performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method are KTUPLE 1, GAP PENALTY=3, WINDOW=5 and DIAGONALS SAVED=5.

A “substantial portion” of an amino acid or nucleotide sequence is that portion comprising enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Basic Local Alignment Search Tool; Altschul, S. F., et al., (1993) J. Mol. Biol. 215:403-410. In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence. The instant specification teaches partial or complete amino acid and nucleotide sequences encoding one or more particular microbial proteins. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

The disclosure encompasses more than the specific exemplary sequences because it is well known in the art that alterations in a gene which result in the production of a chemically equivalent amino acid at a given site, but do not effect the functional properties of the encoded protein are common. For the purposes of the present invention substitutions are defined as exchanges within one of the following five groups: (1) Small aliphatic, nonpolar or slightly polar residues: Ala, Ser, Thr (Pro, Gly); (2) Polar, negatively charged residues and their amides: Asp, Asn, Glu, Gln; (3) Polar, positively charged residues: His, Arg, Lys; (4) Large aliphatic, nonpolar residues: Met, Leu, Ile, Val (Cys); and (5) Large aromatic residues: Phe, Tyr, Trp.

Thus, a codon for the amino acid alanine, a hydrophobic amino acid, may be substituted by a codon encoding another less hydrophobic residue (such as glycine) or a more hydrophobic residue (such as valine, leucine, or isoleucine). Similarly, changes which result in substitution of one negatively charged residue for another (such as aspartic acid for glutamic acid) or one positively charged residue for another (such as lysine for arginine) can also be expected to produce a functionally equivalent product. In many cases, nucleotide changes which result in alteration of the N-terminal and C-terminal portions of the protein molecule would also not be expected to alter the activity of the protein.

The term “complementary” is used to describe the relationship between nucleotide bases that are capable to hybridizing to one another. For example, with respect to DNA, adenosine is complementary to thymine and cytosine is complementary to guanine. Accordingly, the instant invention also includes isolated nucleic acid fragments that are complementary to the complete sequences as reported in the accompanying Sequence Listing as well as those substantially similar nucleic acid sequences.

Suitable nucleic acid fragments not only have the above homologies but typically encode a polypeptide having at least 50 amino acids, preferably at least 100 amino acids, more preferably at least 150 amino acids, still more preferably at least 200 amino acids, and most preferably at least 250 amino acids.

Skilled artisans understand that a sequence can be “codon-optimized” to account for the bias exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

The nuclease module can also comprise a zinc finger nuclease (ZFN) or a transcription activator-like effector nucleases (TALEN), examples of which are well described in the art for purposes including chromosomal mutagenesis and genome editing. See, e.g., PCT Pub. No. WO 2011/091324, US Pat. Pub. Nos. 2012/0017290, 2012/0329067, 2012/0219959, and 2010/0257638.

6.6. Phagemid Construction

Disclosed polynucleotides are typically present within a molecule of bacteriophage genetic material known as a phagemid. Bacteriophages are engineered to produce polypeptides of the disclosure by alteration of phagemids. Techniques for the manipulation of nucleic acids, including techniques for the synthesis, isolation, cloning, detection, and identification are well known in the art and are well described in the scientific and patent literature. See, e.g., Sambrook et al., eds., Molecular Cloning: A Laboratory Manual (2nd Ed.), Vols. 1-3, Cold Spring Harbor Laboratory (1989); Ausubel et al., eds., Current Protocols in Molecular Biology, John Wiley & Sons, Inc., New York (1997); Tijssen, ed., Laboratory Techniques in Biochemistry and Molecular Biology: Hybridization With Nucleic Acid Probes, Part I. Theory and Nucleic Acid Preparation, Elsevier, N.Y. (1993). Nucleic acids comprising the polynucleotides described herein or components thereof include isolated, synthetic, and recombinant nucleic acids.

Engineered phagemids and components thereof can readily be made and manipulated from a variety of sources, either by cloning from genomic or complementary DNA, e.g., by using the well known polymerase chain reaction (PCR). See, for example, Innis et al., 1990, PCR Protocols: A Guide to Methods and Application, Academic Press, New York. Engineered phagemids and components thereof can also be made by chemical synthesis, as described in, e.g., Adams, 1983, J. Am. Chem. Soc. 105:661; Belousov, 1997, Nucleic Acids Res. 25:3440-3444; Frenkel, 1995, Free Radic. Biol. Med. 19:373-380; Blommers, 1994, Biochemistry 33:7886-7896; Narang, 1979, Meth. Enzymol. 68:90; Brown, 1979, Meth. Enzymol. 68:109; Beaucage, 1981, Tetra. Lett. 22:1859; U.S. Pat. No. 4,458,066.

6.7. Production and Purification of Bacteriophages

Disclosed bacteriophages are produced by packaging of genetic material comprising polynucleotides of the disclosure into bacteriophage capsids using known methods. The commercial MaxPlax Lambda Packaging Extract (EPICENTRE Biotechnologies, Madison, Wis.) is a kit that allows packaging of cos site-containing methylated or unmethylated DNA into lambda phage, using two complementary lysogenic E. coli strains, BHB2690 and BHB2688 (see Hohn, E. G., 1979, Methods Enzymol. 68:299). E. coli K-12 strain NM759 can be used in place of strain BHB2690.

Methods for producing and isolating bacteriophages are known. See, e.g., U.S. Pat. Nos. 7,588,929 and 8,105,579.

6.8. Pharmaceutical Compositions

Compositions comprising a bacteriophage of the disclosure and, optionally one or more additional therapeutic agents, such as the second therapeutic agents described in Section 6.9 below, are provided herein. The compositions will usually be supplied as part of a sterile, pharmaceutical composition that will normally include a pharmaceutically acceptable carrier. This composition can be in any suitable form (depending upon the desired method of administering it to a patient).

The bacteriophages of the disclosure can be administered to a patient by a variety of routes such as orally, transdermally, subcutaneously, intranasally, intravenously, intramuscularly, intraocularly, topically, intrathecally and intracerebroventricularly. The most suitable route for administration in any given case will depend on the particular bacteriophage, the subject, and the nature and severity of the disease and the physical condition of the subject.

The effective dose of a bacteriophage of the disclosure can range from about 0.001 to about 75 mg/kg per single (e.g., bolus) administration, multiple administrations or continuous administration, or to achieve a serum concentration of 0.01-5000 mg/ml serum concentration per single (e.g., bolus) administration, multiple administrations or continuous administration, or any effective range or value therein depending on the condition being treated, the route of administration and the age, weight and condition of the subject. In a certain embodiment, each dose can range from about 0.5 mg to about 50 mg per kilogram of body weight, for example from about 3 mg to about 30 mg per kilogram body weight. The bacteriophage can be formulated as an aqueous solution and administered by subcutaneous injection.

Pharmaceutical compositions can be conveniently presented in unit dose forms containing a predetermined amount of a bacteriophage of the disclosure per dose. Such a unit can contain for example but without limitation 5 mg to 5 g, for example 10 mg to 1 g, or 20 to 50 mg. Pharmaceutically acceptable carriers for use in the disclosure can take a wide variety of forms depending, e.g., on the condition to be treated or route of administration.

Therapeutic formulations of the bacteriophages of the disclosure can be prepared for storage as lyophilized formulations or aqueous solutions by mixing the bacteriophage having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives must be nontoxic to the recipients at the dosages and concentrations employed.

Buffering agents help to maintain the pH in the range which approximates physiological conditions. They can be present at concentration ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium gluconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives can be added to retard microbial growth, and can be added in amounts ranging from 0.2%4% (w/v). Suitable preservatives for use with the present disclosure include phenol, benzyl alcohol, meta-cresol, methyl paraben, propyl paraben, octadecyldimethylbenzyl ammonium chloride, benzalkonium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccacharides such as raffinose; and polysaccharides such as dextran. Stabilizers can be present in the range from 0.1 to 10,000 weights per part of weight active protein.

Non-ionic surfactants or detergents (also known as “wetting agents”) can be added to help solubilize the therapeutic agent as well as to protect the therapeutic protein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), Pluronic polyols, polyoxyethylene sorbitan monoethers (TWEEN®-20, TWEEN®-80, etc.). Non-ionic surfactants can be present in a range of about 0.05 mg/ml to about 1.0 mg/ml, for example about 0.07 mg/ml to about 0.2 mg/ml.

Additional miscellaneous excipients include bulking agents (e.g., starch), chelating agents (e.g., EDTA), antioxidants (e.g., ascorbic acid, methionine, vitamin E), and cosolvents.

The formulation herein can also contain a second therapeutic agent in addition to the bacteriophage of the disclosure. Examples of suitable second therapeutic agents are provided in Section 6.9 below.

The dosing schedule for subcutaneous administration can vary from once a month to daily depending on a number of clinical factors, including the type of disease, severity of disease, and the patient's sensitivity to the bacteriophage.

The dosage of a bacteriophage of the disclosure to be administered will vary according to the particular bacteriophage, the type of autoimmune or inflammatory disease, the subject, and the nature and severity of the disease, the physical condition of the subject, the therapeutic regimen (e.g., whether a second therapeutic agent is used), and the selected route of administration; the appropriate dosage can be readily determined by a person skilled in the art.

For the treatment and/or prophylaxis of autoimmune or inflammatory disease in humans and animals, pharmaceutical compositions comprising bacteriophagex can be administered to patients (e.g., human subjects) at therapeutically or prophylactically effective dosages (e.g., dosages which result in inhibition of an autoimmune or inflammatory disease and/or relief of autoimmune or inflammatory disease symptoms) using any suitable route of administration, such as injection and other routes of administration known in the art.

It will be recognized by one of skill in the art that the optimal quantity and spacing of individual dosages of a bacteriophage of the disclosure will be determined by the nature and extent of the condition being treated, the form, route and site of administration, and the age and condition of the particular subject being treated, and that a physician will ultimately determine appropriate dosages to be used. This dosage can be repeated as often as appropriate. If side effects develop the amount and/or frequency of the dosage can be altered or reduced, in accordance with normal clinical practice.

Pharmaceutical compositions comprising a disclosed bacteriophage can be formulated in a unit dose or multi-dose formulation. Suitable formulations can be selected from the group consisting of ointments, solutions, suspensions or emulsions, extracts, powders, granules, sprays, lozenges, tablets or capsules and additionally include a dispersing agent or a stabilizing agent.

6.9. Combination Therapy

Described below are combinatorial methods in which the bacteriophages of the disclosure can be utilized. The combinatorial methods of the disclosure involve the administration of at least two agents to a patient, the first of which is a bacteriophage of the disclosure, and the second of which is a second therapeutic agent. The bacteriophage and the second therapeutic agent can be administered simultaneously, sequentially or separately.

The combinatorial therapy methods of the present disclosure can result in a greater than additive effect, providing therapeutic benefits where neither the bacteriophage or second therapeutic agent administered in an amount that is alone therapeutically effective.

In the present methods, the bacteriophage of the disclosure and the second therapeutic agent can be administered concurrently, either simultaneously or successively. As used herein, the bacteriophage of the disclosure and the second therapeutic agent are said to be administered successively if they are administered to the patient on the same day, for example during the same patient visit. Successive administration can occur 1, 2, 3, 4, 5, 6, 7 or 8 hours apart. In contrast, the bacteriophage of the disclosure and the second therapeutic agent are said to be administered separately if they are administered to the patient on the different days, for example, the bacteriophage of the disclosure and the second therapeutic agent can be administered at a 1-day, 2-day or 3-day, one-week, 2-week or monthly intervals. In the methods of the present disclosure, administration of the bacteriophage of the disclosure can precede or follow administration of the second therapeutic agent.

As a non-limiting example, the bacteriophage of the disclosure and second therapeutic agent can be administered concurrently for a period of time, followed by a second period of time in which the administration of the bacteriophage of the disclosure and the second therapeutic agent is alternated.

Because of the potentially synergistic effects of administering a bacteriophage of the disclosure and a second therapeutic agent, such agents can be administered in amounts that, if one or both of the agents is administered alone, is/are not therapeutically effective.

In certain aspects, the second therapeutic agent is an antibiotic, an anti-inflammatory agent, an immunosuppressive agent, a local anesthetic agent, or a corticosteroid.

The antibiotic is typically selected from: amikacin, gentamicin, kanamycin, neomycin, netilmicin, paromomycin, rhodostreptomycin, streptomycin, tobramycin, apramycin, rifamycin, naphthomycin, mupirocin, geldanamycin, ansamitocin, carbacephems, imipenem, meropenem, ertapenem, faropenem, doripenem, panipenem/betamipron, biapenem, PZ-601, cephalosporins, cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefonicid, cefprozil, cefuroxime, cefuzonam, cefinetazole, cefotetan, cefoxitin, cefcapene, cefdaloxime, cefdinir, cefditoren, cefetamet, cefixime, cefmenoxime, cefteram, ceftibuten, ceftiofur, ceftiolene, ceftizoxime, ceftriaxone, cefoperazone, ceftazidime latamoxef, cefclidine, cefepime, cefluprenam, cefoselis, cefozopran, cefpirome, cefquinome, flomoxef, ceftobiprole, azithromycin, clarithromycin, dirithromycin, erythromycin, roxithromycin, aztreonam, pencillin and penicillin derivatives, actinomycin, bacitracin, colistin, polymyxin B, cinoxacin, flumequine, nalidixic acid, oxolinic acid, piromidic acide, pipemidic acid, rosoxacin, ciprofloxacin, enoxacin, fleroxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, garenoxacin, gemifloxacin, stifloxacin, trovalfloxacin, prulifloxacin, acetazolamide, benzolamide, bumetanide, celecoxib, chlorthalidone, clopamide, dichlorphenamide, dorzolamide, ethoxyzolamide, furosemide, hydrochlorothiazide, indapamide, mafendide, mefruside, metolazone, probenecid, sulfacetamide, sulfadimethoxine, sulfadoxine, sulfanilamides, sulfamethoxazole, sulfasalazine, sultiame, sumatriptan, xipamide, tetracycline, chlortetracycline, oxytetracycline, doxycycline, lymecycline, meclocycline, methacycline, minocycline, and rolitetracycline.

Anti-inflammatory agents include, but are not limited to, dexamethasone, pentasa, mesalazine, asacol, codeine phosphate, benorylate, fenbufen, naprosyn, diclofenac, etodolac and indomethacin, aspirin and ibuprofen.

Local anesthetics that may be used in pharmaceutical compositions of the present invention include tetracaine, tetracaine hydrochloride, lidocain, lidocaine hydrochloride, dimethisoquin hydrochloride, dibucaine, dibucaine hydrochloride, butambenpicrate, and pramoxine hydrochloride. An exemplary concentration of local anesthetic is about 0.025% to about 5% by weight of the total composition.

Corticosteroids that may be useful include betamethasone, dipropionate, fluocinolone, actinide, betamethasone valerate, triamcinolone actinide, clobetasol propionate, desoximetasone, diflorasone diacetate, amcinonide, flurandrenolide, hydrocortisone valerate, hydrocortisone butyrate, and desonide. An exemplary concentration of corticosteroid is about 0.01% to about 1% by weight of the total composition.

6.10. Antiseptic Compositions

The compositions provided herein may also be used as sterilizing or cleaning aids for use, for example, on surfaces to reduce and/or eliminate contamination by MRSA, VISA, VRSA or VRE. For example, compositions of the present invention may be added to or diluted in an appropriate excipient or solution prior to use as a sterilizing or cleaning agent. Exemplary excipients are described above. Such sterilizing or cleaning solutions may be used to decontaminate, for example, furniture, floors, equipment including for example specialized hospital equipment and/or surgical equipment. Disclosed bacteriophage compositions may be diluted as an aqueous or non-aqueous solution (dissolved in aqueous, non aqueous or organic solvent) and which may be applied to a body part, for example the hands. Such a solution may find particular application in, for example hospitals, care homes and or nurseries where the prevalence and transmission rates of MRSA, VISA, VRSA or VRE are often high.

6.11. Methods of Treatment

The disclosure provides methods of treating a disorder or disease caused by a bacterial infection comprising administering a pharmaceutical composition comprising a disclosed bacteriophage.

Non-limiting examples of disorders/diseases caused by bacterial infections or toxins produced during bacterial infections, and for which the compositions and methods described herein are applicable in various aspects and embodiments, include, but are not limited to, pneumonia, sepsis or bacteremia, toxic shock syndrome, bacterial meningitis, endocarditis, gastroenteritis, peritonitis, strep throat, osteomyelitis, cholera, diphtheria, tuberculosis, anthrax, botulism, brucellosis, campylobacteriosis, typhus, ear infections (e.g., otitis media), including recurrent ear infections, recurrent pneumonia, gonorrhea, hemolytic-uremic syndrome, listeriosis, lyme disease, mastitis, peritonitis, rheumatic fever, pertussis (Whooping Cough), plague, salmonellosis, scarlet fever, shigellosis, sinusitis, including chronic sinusitis, syphilis, trachoma, tularemia, typhoid fever, and urinary tract infections, including chronic urinary tract infections. In other embodiments, the disorder or disease is an infection of soft tissue or skin, such as acne, cellulitis, abscess, necrotizing fasciitis, impetigo, erysipelas, or an infection of a burn or wound, including surgical wounds and skin ulcer (e.g., diabetic ulcer).

The compositions of the present disclosure may be used in methods of preventing and treating infections associated with E. faecalis, E. faecium, P. aeruginosa, S. aureus, and/or A. baumannii in both humans and animals. In other embodiments, the compositions of the present invention may be used to treat infection associated with related species or strains of these bacteria, including, but not limited to S. epidermidis, S. auricularis, S. capitis, S. haemolyticus, S. hominis, S. saprophyticus, S. simulans, S. xylosis, Micrococcus luteus, Bacillus subtilis, B. pumilus, E. hirae, and/or one or more of the strains of A. baumannii.

In specific embodiments, the subject receiving a pharmaceutical composition of the invention is a mammal (e.g., bovine, ovine, caprine, equid, primate (e.g., human), rodent, lagomorph or avian (e.g., chicken, duck, goose)). In the context of the present invention, “treatment” refers to therapeutic treatment and wherein the object is to eliminate, lessen, decrease the severity of, ameliorate, slow the progression of or prevent the symptoms or underlying cause (e.g., bacterial infection) associated with the pathological condition or disorder. “Treatment” refers to both therapeutic treatment and prophylactic or preventative measures, wherein the object is to eliminate, lessen, decrease the severity of, slow the progression of or delay or prevent the symptoms or underlying cause (e.g., bacterial infection) associated with the pathological condition or disorder. It is also contemplated that a disclosed composition acts as a prophylactic or preventative measure, preventing the onset of infection caused by one or more bacteria.

A. baumannii, E. faecalis, E. faecium, P. aeruginosa and S. aureus are responsible for many severe opportunistic infections, particularly in individuals with compromised immune systems. The pharmaceutical compositions of the present invention are contemplated for treating any infection associated with A. baumannii, E. faecalis, E. faecium, P. aeruginosa or S. aureus, or associated with other species or strains of bacteria, including, but not limited to, infections of the skin (including but not limited to skin ulcers, bed sores and diabetic foot ulcers), infections in and around wounds, post-operative infections, infections associated with catheters and surgical drains and infections of the blood.

A. baumannii, E. faecalis, E. faecium, P. aeruginosa and S. aureus are also associated with infections that involve organ systems that have a high fluid content, and it is contemplated that the disclosed pharmaceutical compositions have therapeutic use in the prevention and treatment of these infections. For example, the pharmaceutical compositions may be used for the prevention or treatment of infections of the respiratory tract, of the cerebrospinal fluid, of peritoneal fluid, and of the urinary tract. The compositions of the invention may also be used to prevent and/or treat nosocomial pneumonia, infections associated with continuous ambulatory peritoneal dialysis (CAPD), catheter-associated bacteruria, and nosocomial meningitis.

In a preferred embodiment, a disclosed bacteriophage is used prophylactically in hospital setting, particularly to prevent infections associated with wounds, ulcers, and openings in the skin due to catheterization, and any other medical procedures or devices.

In certain embodiments, a disclosed bacteriophage is used as a single agent for treating or preventing infections associated with A. baumannii, E. faecalis, E. faecium, P. aeruginosa, S. aureus or other bacterial species. In other embodiments of the invention, a disclosed pharmaceutical composition is used in combination with other agents, such as those described in Section 6.9.

The pharmaceutical compositions of the invention can be administered by inhalation, in the form of a suppository or pessary, topically (e.g., in the form of a lotion, solution, cream, ointment or dusting powder), epi- or transdermally (e.g., by use of a skin patch), orally (e.g., as a tablet, which may contain excipients such as starch or lactose), as a capsule, ovule, elixirs, solutions or suspensions (each optionally containing flavoring, coloring agents and/or excipients), or they can be injected parenterally (e.g., intravenously, intramuscularly or subcutaneously). For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner. In a preferred embodiment, a bacteriophage of the present invention is administered topically, either as a single agent, or in combination with other antibiotic treatments as described herein or known in the art.

A pharmaceutical composition may also be dermally or transdermally administered. For topical application to the skin, the pharmaceutical composition may be combined with one, or a combination of carriers, which include but are not limited to, an aqueous liquid, an alcohol base liquid, a water soluble gel, a lotion, an ointment, a nonaqueous liquid base, a mineral oil base, a blend of mineral oil and petrolatum, lanolin, liposomes, proteins carriers such as serum albumin or gelatin, powdered cellulose carmel, and combination thereof. A topical mode of delivery may include a smear, a spray, a time-release patch, a liquid absorbed wipe, and combinations thereof. A disclosed bacteriophage may be applied to a patch or bandage either directly or in one of the carriers. The patches may be damp or dry, wherein the pharmaceutical composition is in a lyophilized form on the patch. The carriers of topical compositions may comprise semi-solid and gel-like vehicles that include a polymer thickener, water, preservatives, active surfactants, or emulsifiers, antioxidants, sun screens, and a solvent or mixed solvent system. U.S. Pat. No. 5,863,560 discloses a number of different carrier combinations that can aid in the exposure of skin to a medicament, and its contents are incorporated herein.

For intranasal or administration by inhalation, pharmaceutical composition of the invention is conveniently delivered in the form of a dry powder inhaler or an aerosol spray presentation from a pressurized container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurized aerosol, the dosage unit may be determined by providing a valve to deliver a metered amount. The pressurized container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the pharmaceutical composition and a suitable powder base such as lactose or starch.

For administration in the form of a suppository or pessary, the therapeutic compositions may be applied topically in the form of a gel, hydrogel, lotion, solution, cream, ointment or dusting powder. Compositions of the invention may also be administered by the ocular route. For ophthalmic use, the compositions of the invention can be formulated as micronized suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

Dosages and desired drug concentrations of the pharmaceutical compositions of the present invention may vary depending on the particular use. The determination of the appropriate dosage or route of administration is well within the skill of an ordinary physician. Animal experiments can provide reliable guidance for the determination of effective doses in human therapy. Interspecies scaling of effective doses can be performed by one of ordinary skill in the art following the principles described by Mordenti, J. and Chappell, W. “The use of interspecies scaling in toxicokinetics” in Toxicokinetics and New Drug Development, Yacobi et al., Eds., Pergamon Press, New York 1989, pp 42-96.

6.12. Methods of Killing Pathogenic Bacterial Host Cells or Restricting their Growth

Bacteriophages of the disclosure comprise a polynucleotide and bind to specific bacterial host cells, thereby contacting genetic material within the host cell with the polynucleotide. In some embodiments, the polynucleotide comprises a targeting module and an endonuclease. The targeting module tethers the endonuclease to a specific target DNA sequence, producing a double-strand break at the target DNA sequence. A plurality of double-strand breaks in the genome of a host organism is known to cause the host cell to die, or to restrict its ability to replicate and divide (i.e., to grow properly). Accordingly, in some embodiments, a plurality of bacteriophages targets and introduces a plurality of double-strand breaks at a plurality of target DNA sequences within a host cell, thereby killing the host cell. In some embodiments, a plurality of bacteriophages targets and introduces a plurality of double-strand breaks at a plurality of target DNA sequences within a host cell, thereby restricting growth of the host cell.

The disclosure provides methods of killing a host cell and/or restricting the growth of a host cell by contacting the host cell with a bacteriophage of the disclosure.

6.13. Methods of Decreasing Bacterial Drug Resistance

As described in Section 6.3, a target DNA sequence can be located within a gene that confers resistance to a drug agent. Introduction of double-strand breaks within a drug resistance gene will result in loss of resistance by the host cell to the drug.

Thus, the disclosure provide methods of decreasing bacterial drug resistance by contacting the host cell with a bacteriophage of the disclosure.

6.14. Methods of Engineering Microbiomes

Skilled artisans will appreciate that the specificity of the disclosed methods with respect to targeting of host cells of specific strains allows for use of the methods in microbiome engineering. In some embodiments, a sequence specific to a particular bacterial strain (e.g., 16S ribosomal RNA sequence) is the target DNA sequence. Thus, methods are provided for modulating one or more specific strains of bacteria within a heterogenous population of bacteria (e.g., the gut or skin microbiome of a human host). In modulating the one or more specific strains of bacteria within a heterogenous population of bacteria, it is meant that the relative representation of the one more specific strains within the population is increased or decreased.

### 7. EXAMPLES

### Example 1

### Vector Construction

A plasmid containing a Cas9 targeted nuclease transcriptionally driven by a host-compatible promoter was introduced into E. coli via bacteriophage transduction. This plasmid backbone contains COS sites to allow for bacteriophage packaging and transduction. The plasmid contains a tracrRNA which mediates the interaction between Cas9 and a guide RNA. The guide RNA in this example is a nucleotide sequence that is homologous to a beta-lactam resistance gene (bla) which confers resistance to ampicillin in this target organism (E. coli). This sequence can be of any form as long as it is compatible with protospacer adjacent motif (PAM) requirements of different Cas9 homologues.

(5′-NNNNNNNNNNNN-NG-3 (SpCas9 PAM)
5′-NNNNNNNNNNNN-CCN-3 (H. solfataricus)
5′-NNNNNNNNNNNN-TCN-3 (H. solfataricus)
5′-NNNNNNNNNNNN-TTC-3 (H. walsbyi)
5′-NNNNNNNNNNNN-AWG-3 (E. coli)
5′-NNNNNNNNNNNN-CC-3 (P. aeruginosa)
5′-NNNNNNNNNNNN-NNAGAAW-3 (S. thermophilus)
5′-NNNNNNNNNNNN-NGGNG-3 (S. thermophilus)
5′-NNNNNNNNNNNN-GATT-3 (N. meningititus))

In this specific example, we chose a guide RNA that is compatible with the SpCas9 PAM in the form of 5′-aaaccgcctccatccagtctattaattgttgccg-3′. This sequence targets a portion of the bla gene found in pUC19 as a demonstration of immunization against incoming plasmid and foreign DNA that contains a beta lactamase or gene containing the same guide sequence. We utilized lambda phage, which is able to infect E. coli, as the delivery bacteriophage.

The plasmid containing Cas9, tracrRNA, and the bla guide RNA was ligated to a fragment of pWEB™ (Epicentre Biotechnologies) cosmid vector backbone containing COS sites for lambda phage packaging. The elements in the vector each allow for bacteriophage packaging (COS), propagation in E. coli for engineering (SV40 and p15A origins, chloramphenicol resistance cassette), Cas9 expression in the target host, tracr RNA transcription in the target host, and guide RNA transcription in the target host. Alternatively, a hybrid tracr-guideRNA is used to mediate gene targeting as reported in the art in other forms. The complete construct containing all of these elements is linearized, dephosphorylated using T4 polynucleotide kinase, and ligated to randomly sheared stuffer DNA to provide an optimal substrate for bacteriophage packaging as different bacteriophage will efficiently package DNA of specific sizes only. In this demonstration the optimal overall construct size is ˜45 kb. Lambda phage packaging extract (MaxPlax Lambda Packaging Extracts™, Epicentre Biotechnologies) was used to package the ligated construct DNA to form circular plasmids which can be efficiently transduced into E. coli.

### Example 2

### Lambda Phage Packaging, Transduction and Targeted Ampicillin Gene Disruption

E. coli was transduced with packaged bacteriophage and cosmid DNA to yield recombinant bacteria containing the immunization plasmid (see Example 1). The plasmid was maintained using Luria Broth (LB) supplemented with chloramphenicol at 12.5 μg/mL.

A secondary infection of the bacterium was mediated by introducing a plasmid, pUC19, with an origin of replication that is compatible with the immunization construct, and which also contained a bla resistance gene. First, competent cells containing the immunization construct were prepared using published methods. pUC19 DNA was introduced into competent cells via chemical transformation and relative efficiencies of pUC19 transformation were compared between negative control immunization constructs which containing no guide sequences and those containing bla-specific guide sequences outlined above.

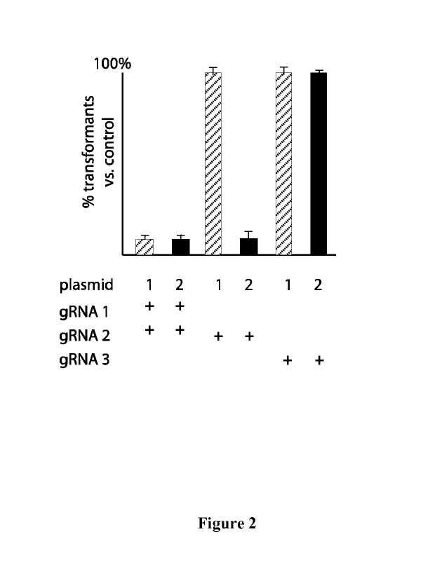

With negative control immunization constructs containing no guide RNA sequence, pUC19 was transformed into the target strain at a frequency of >10⁶ transformants per microgram of plasmid DNA. With immunization constructs containing the bla-specific guide sequence, no transformants were successfully generated, representing a clear demonstration of bacterial immunity to transformation with a plasmid containing antibiotic resistance plasmid. Thus, the bacteriophage delivery system was successfully used to introduce a CRISPR-Cas9 targeting system to immunize a bacterium against foreign DNA introduction (See FIG. 2).

Plasmids containing one of two genes encoding resistance to ampicillin (plasmid-1) or kanamycin (plasmid-2) were stably introduced into E. coli. Next, a construct expressing a guide RNA cassette aimed at plasmids 1+2 (multiplex), plasmid 2 alone, or a non-homologous sequence (gRNA-3) was introduced into the recombinant strains. Doubly-recombinant strains containing the initial resistance gene-encoding plasmid and the guide RNA plasmid were selected using a third antibiotic marker (chloramphenicol) and briefly incubated to allow for transcription and processing of the guide RNAs. Transformants were then plated on selective media containing either ampicillin+chloramphenicol, or kanamycin+chloramphenicol and colony counts were compared vs. controls plated on chloramphenicol alone. The percentage of recovered recombinant strains with intact resistance genes was then compared.

### Example 3

### Targeted Genome Disruption

A similar approach as in Examples 1 and 2 is used to target existing genetic sequences (carried on chromosomes or plasmids) contained within a target organisms of interest. The order of plasmid introduction, in this case, is reversed to demonstrate the targeted cleavage or engineering of specific DNA sequences contained within a microbe. In this demonstration, pUC19 is introduced into chemically competent E. coli using established techniques. Transformed colonies are isolated and competent cells prepared from these cells for bacteriophage transduction. Next, packaged bacteriophage containing a targeting construct, comprised of a Cas9 expression cassette, guide and tracr RNA (either in separate transcriptional units or fused as a hybrid tracr/guide RNA) is used to transduce the bacteria containing the plasmid (pUC19 carrying a bla resistance cassette). Control plasmids that lack a bla-specific guide sequence are transduced in parallel to assess the efficiency of targeting. The transduced recombinants are plated on LB-agar containing chloramphenicol, ampicillin, or both antibiotics. In control constructs, which lack the appropriate guide sequence, multiple transformants could be isolated. In constructs containing a guide sequence targeting bla, colonies could be recovered only from those plates containing chloramphenicol to select for the targeting plasmid alone. These results indicate that the targeting construct is cleaving and destroying the bla-containing pUC19 plasmid.

This strategy can be applied to any number of bacteriophage compatible bacterium that harbor plasmids containing virtually any DNA sequence of interest. In some embodiments, this plasmid can harbor metallobetalactamases responsible for therapeutic antibiotic resistance such as CRE (carbapenem resistant enterobactericeae) or Klebsiella pneumonia. In other embodiments, the target sequence (guide sequence) can be located in the genome of the target organism, as opposed to a plasmid, to engineer or kill the host strain. In other embodiments, multiple guide sequences can be used to target multiple locations in the bacterial genome or DNA sequence of interest.

### Example 4

### Microbiome Engineering with CRISPR Systems

In this example a specific strain of bacterium contained within a complex microbiome, or collection of microorganisms, is targeted for specific CRISPR-engineering by utilizing strain-specific guide sequences. In this demonstration, two strains of E. coli, DH10B and EPI300, are co-cultured in LB media at a defined ratio. A lambda phage packaging reaction containing the a CRISPR-Cas9 expression construct, tracrRNA, and a guide RNA sequence targeting trfA are used to transduce the co-cultured strains and resulting constructs are plated on LB agar. Transduced recombinant colonies were analyzed (by sequencing PCR products specific for trfA and other strain specific markers) to determine the relative proportion of bacterium that are genotypically EPI300 or DH10B. Transduction with a control CRISPR-Cas9 construct lacking a guide RNA shows no alteration in the ratio of the two strains whereas transduction with a construct containing the trfA guideRNA eliminated the EPI300 strain from the resulting co-culture. These results show that, although both strains are transduced, the strain which contains a target for the guideRNA in the CRISPR-Cas9 expression construct (in this demonstration trfA) is eliminated from the culture.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

1-13. (canceled)

14. A bacteriophage comprising a polynucleotide that expresses:
(a) an RNA-directed DNA-binding polypeptide comprising a nuclease module; and
(b) a targeting module comprising a guide RNA,
wherein the targeting module tethers the RNA-directed DNA-binding polypeptide to a target DNA sequence within a prokaryotic host cell, thereby producing a double-strand break within the target sequence.

15. The bacteriophage of claim 14, wherein the prokaryotic host cell is an antibiotic-resistant host cell and the target DNA sequence is within a gene that confers resistance to said antibiotic.

16. The bacteriophage of claim 15, wherein the prokaryotic host cell is of a species selected from: Escherichia coli, Acinetobacter baumannii, Enterococcus faecalis, Enterococcus faecium, Pseudomonas aeruginosa, Staphylococcus aureus, Staphylococcus epidermidis, Staphylococcus auricularis, Staphylococcus capitis, Staphylococcus haemolyticus, Staphylococcus hominis, Staphylococcus saprophyticus, Staphylococcus simulans, Staphylococcus xylosis, Micrococcus luteus, Bacillus subtilis, Bacillus pumilus, Enterococcus hirae, Enterococcus avium, and Klebsiella pneumonia.

17. The bacteriophage of claim 16, wherein the prokaryotic host cell is an Escherichia coli cell, and the antibiotic is ampicillin.

18. The bacteriophage of claim 16, wherein the prokaryotic host cell is a Staphylococcus aureus cell, and the antibiotic is methicillin.

19. The bacteriophage of claim 14, wherein the polynucleotide comprises:
(a) a nucleic acid sequence encoding the amino acid sequence as set forth in SEQ ID NO:2;
(b) a nucleic acid molecule that hybridizes with (a) under the following hybridization conditions: 0.1×SSC, 0.1% SDS, 65° C. and washed with 2×SSC, 0.1% SDS followed by 0.1×SSC, 0.1% SDS; or
(c) an isolated nucleic acid molecule that is complementary to (a) or (b).

20. The bacteriophage of claim 19, wherein the polynucleotide comprises a nucleic acid sequence encoding the amino acid sequence as set forth in SEQ ID NO:2.

21. A pharmaceutical composition, comprising a bacteriophage according to claim 14 and a pharmaceutically acceptable carrier.

22. An antiseptic composition, comprising a bacteriophage according to claim 14 and an excipient.

23. A method of restricting growth of a host cell, comprising contacting the host cell to a bacteriophage, where in the bacteriophage specifically binds to the host cell and introduces a polynucleotide into the host cell, wherein the polynucleotide encodes:
(i) an RNA-directed DNA-binding polypeptide comprising a nuclease module; and
(ii) a targeting module comprising a guide RNA;
wherein the introduction of the polynucleotide into the host cell induces the expression of the RNA-directed DNA-binding polypeptide and the targeting module within the host cell, and wherein the targeting module directs the RNA-directed DNA-binding polypeptide to a target DNA sequence within the host cell, thereby introducing a double-strand break at the target DNA sequence and restricting growth of the host cell.

24. The method of claim 23, wherein the target DNA sequence is within a gene conferring antibiotic resistance to the host cell.

25. The method of claim 24, wherein the target DNA sequence is within an antibiotic resistance gene carried on a plasmid or episomal vector, thereby sensitizing the host cell to antibiotic treatment.

26. The method of claim 23, wherein the host cell is contacted to a plurality of bacteriophages, where each bacteriophage specifically binds to the host cell and introduces a polynucleotide into the cell,
wherein each polynucleotide encodes: (a) an RNA-directed DNA-binding polypeptide comprising a nuclease module; and (b) a targeting module comprising a guide RNA,
wherein each guide RNA specifically directs an RNA-directed DNA-binding polypeptide to one of a plurality of DNA sequences within the host cell, thereby introducing a plurality of double-strand breaks within the plurality of DNA sequences within the host cell,
thereby killing the host cell.

27. A method of decreasing the relative representation of a specific strain of bacteria within a heterogenous population of bacteria, comprising contacting the heterogenous population of bacteria with a bacteriophage comprising a polynucleotide that expresses
(a) an RNA-directed DNA-binding polypeptide comprising a nuclease module; and
(b) a targeting module comprising a guide RNA,
wherein the targeting module tethers the RNA-directed DNA-binding polypeptide to a target DNA sequence within, thereby producing a double-strand break within the target sequence,
wherein the target sequence is unique to the specific strain of bacteria, among the heterogenous population of bacteria.


SEQUENCE LISTING (3 sequences)

SEQ ID NO: 1 - DNA, 34 bp, Escherichia coli
aaaccgcctccatccagtctattaattgttgccg

SEQ ID NO: 2 - protein, 1368 aa, Streptococcus pyogenes [sequence omitted]

SEQ ID NO: 3 - DNA, 4107 bp, Streptococcus pyogenes
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga